(19) United States
(12) Patent Application Publication
(10) Pub. No.: US 2020/0222305 A1
(43) Pub. Date: Jul. 16, 2020

(54) NATURAL SKIN CARE COMPOSITION

(71) Applicant: Shellie Ann Black, Alpine, UT (US)

(72) Inventor: Shellie Ann Black, Alpine, UT (US)

(21) Appl. No.: 16/249,150

(22) Filed: Jan. 16, 2019

(51) Int. Cl. A61K 8/9789; A61K 8/34; A61K 8/31; A61Q 19/00

(52) U.S. Cl. CPC A61K 8/9789; A61K 8/345; A61K 2800/5922; A61Q 19/007; A61K 8/31

(57) ABSTRACT

A skin care composition formulated entirely with natural ingredients is disclosed. The appropriate ratio of the ingredients provides a semi-solid moisturizer cream that could adhere to the skin for long duration to produce the desired result. The skin care composition comprises a shea extract, a cocoa extract, a glycerin extract, a squalane extract, a copaiba extract, a frankincense extract, a helichrysum extract, a lavender extract and a sandalwood extract. Further, the skin care composition comprises 72.84% of shea extract, 12.35% of cocoa extract, 12.35% to 24.69% of glycerin extract, 0.41% of squalane extract, 0.41% of copaiba extract, 0.41% of frankincense extract, 0.41% of helichrysum extract, 0.41% to 2.47% of lavender extract, and 27.16% to 100% of sandalwood extract. The present invention provides a supple, soft, and younger looking skin naturally, without any artificial ingredients, including binders, fillers, dyes, parabens, fragrances or other harmful substances.

### BACKGROUND OF THE INVENTION

### A. Technical Field

The present invention generally relates to composition for skin care, and more specifically relates to a skin care composition formulated entirely with natural ingredients.

### B. Description of Related Art

Human skin is extremely susceptible to the temperature and humidity extremes of our environment. In addition to environmental factors, skin may also face adverse conditions in the workplace where excessive temperatures or low humidity may harm skin. Exposure to chemicals may also remove moisture from the skin causing damage and actual skin chafing and loss if not treated properly. To counteract adverse environmental conditions and to remain healthy and beautiful under a variety of extreme environmental conditions skin care products are used for skin cleansing and moisturizing, which are addressed to anything from relieving dry skin to allegedly removing wrinkles.

Many of these products claim to be hypoallergenic and non-irritating to most users, however, these products nonetheless contain ingredients that can be irritating or even harmful to individuals who, for some reason, have become highly sensitized to certain chemicals in the skin care products. Few existing compositions aimed to nourish skin is discussed as follows.

US20040057924 relates to a holistic dermatological cleanser. The skin care composition comprises a waters mixture comprising; distilled water and aloe vera mixed in a ratio of about 2:1; vegetable glycerin is added to the distilled water and aloe vera mixture in a ratio of about 1:8; and at least one of an herb and flower chosen from a group of aromatic therapeutic herbs and flowers; an oils mixture comprising a natural plant extract oil is mixed with approximately equal amounts of kokum butter and shea butter in a ratio of about 3:1; a desired amount of cocoa butter; and an amount of emulsifying wax in a range of about 1:8; the oils mixture being combined at a temperature of about 110°-150° F., blended to a slurry and cooled to room temperature wherein the waters mixture is combined with the oils mixture and blended to produce the skin care composition. Even though, this skin care composition only comprises naturally grown organic plant ingredients, this composition lacks to provide desired results in reconditioning and regulating skin conditions defeating the basic purpose of cleanser.

US20120276030 relates to a cosmetic composition. The anhydrous colored cosmetic composition comprises a blend or mixture of a solid butter and a colorant. The anhydrous colored cosmetic composition may also include at least one oil component in addition to the solid butter and colorant. The cosmetic composition does not contain any added exogenous water. Further provides a process for preparing an anhydrous colored cosmetic composition comprising blending of at least one solid butter and a colorant together greater than 50% by weight, wherein no exogenous water is added during the blending.

U.S. Pat. No. 8,623,335 relates to a scar and rosacea and other skin care treatment composition and method. The composition comprises a skin toner for cleansing a skin surface, removing dead skin cells, restoring alkali balance, and shrinking skin pores; and a skin moisturizer for increasing water content in the external layers of the skin. The composition further comprises ingredients such as cocoa butter, shea butter, vegetable glycerin, squalane oil, copaiba oil, frankincense oil, helichrysum oil, lavender oil and sandalwood oil. However, the above described cosmetic composition and skin care treatment composition utilizes chemical compounds.

Therefore, there is need for a skin care composition formulated entirely or at least predominantly with natural ingredients. Further, there is a need for a skin care composition formulated of natural ingredients of appropriate quantity/ration to provide excellent nourishing to skin.

### SUMMARY OF THE INVENTION

The present invention provides a skin care composition formulated entirely with natural ingredients. The skin care composition comprises natural ingredients at appropriate ratio to provide a semi-solid moisturizer cream that could adhere to the skin for long duration to produce the desired result. The present invention provides a supple, soft, and younger looking skin naturally, without any artificial or synthetic ingredients, including binders, fillers, dyes, parabens, fragrances or other harmful substances.

In one embodiment, the skin care composition comprises a shea extract, a cocoa extract, a glycerin extract, a squalane extract, a copaiba extract, a frankincense extract, a helichrysum extract, a lavender extract and a sandalwood extract. In an embodiment, the shea extract is a shea butter. In an embodiment, the cocoa extract is a cocoa butter. In an embodiment, the glycerin extract is a vegetable glycerin. In an embodiment, the squalane extract is a squalene oil. In an embodiment, the copaiba extract is a copaiba oil. In an embodiment, the frankincense extract is a frankincense oil. In an embodiment, the helichrysum extract is a helichrysum oil. In an embodiment, the lavender extract is a lavender oil. In an embodiment, the sandalwood extract is a sandalwood oil.

In one embodiment, the skin care composition comprises 70% to 75% of shea extract, 10%-15% of cocoa extract, 10% to 55% of glycerin extract, 0.40% to 0.45% of squalane extract, 0.40% to 0.45% of copaiba extract, 0.40% to 0.45% of frankincense extract, 0.40% to 0.45% of helichrysum extract, 0.40% to 3% of lavender extract, and 25% to 100% of sandalwood extract.

In another embodiment, the skin care composition comprises 72.84% of shea extract, 12.35% of cocoa extract, 12.35% to 24.69% of glycerin extract, 0.41% of squalane extract, 0.41% of copaiba extract, 0.41% of frankincense extract, 0.41% of helichrysum extract, 0.41% to 2.47% of lavender extract, and 27.16% to 100% of sandalwood extract.

In an embodiment, a method of preparing the composition is disclosed. At one step, the cocoa extract and the shea extract are mixed and heated to a certain temperature. At another step, one or more ingredients are added to the heated cocoa extract and shea extract to form a mixture, wherein the ingredients include glycerin extract, squalane extract, copaiba extract, frankincense extract, helichrysum extract, lavender extract and sandalwood extract. In one embodiment, the ingredients are added at a quantity till the mixture reaches a particular consistency or viscosity. At another step, the mixture is cooled to form the semi-solid skin care composition. Then, the semi-solid skin care composition could be packed, stored and utilized as a moisture cream. The semi-solid composition changes into a thick liquid on application to the skin surface due to the skin temperature. The solid to liquid phase change enables the composition to adhere to the skin for long duration producing the desired or excellent result.

Other objects, features and advantages of the present invention will become apparent from the following detailed description. It should be understood, however, that the detailed description and the specific examples, while indicating specific embodiments of the invention, are given by way of illustration only, since various changes and modifications within the spirit and scope of the invention will become apparent to those skilled in the art from this detailed description.

### BRIEF DESCRIPTION OF DRAWINGS

The embodiments herein will be better understood from the following detailed description with reference to the drawings, in which:

FIG. 1 is a flowchart illustrating a method of producing a skin care composition, according to one embodiment of the present invention.

### DETAILED DESCRIPTION OF EMBODIMENTS

A description of embodiments of the present invention will now be given with reference to the Figures. It is expected that the present invention may be embodied in other specific forms without departing from its spirit or essential characteristics. The described embodiments are to be considered in all respects only as illustrative and not restrictive. The scope of the invention is, therefore, indicated by the appended claims rather than by the foregoing description. All changes that come within the meaning and range of equivalency of the claims are to be embraced within their scope.

The present invention provides a skin care composition formulated entirely or at least predominantly with natural ingredients. The appropriate ratio of the ingredients provides a semi-solid moisturizer cream that could adhere to the skin for long duration to produce the desired result.

In one embodiment, the skin care composition comprises a shea extract, a cocoa extract, a glycerin extract, a squalane extract, a copaiba extract, a frankincense extract, a helichrysum extract, a lavender extract and a sandalwood extract.

In an embodiment, the shea extract is a shea butter. The shea butter has many anti-aging properties and vitamin A. The shea butter has high levels of oleic, linoleic, and stearic acids. These acids are an effective ingredient to combat oxidative stress, which is the effect of environmental toxins on skin cells. This helps to have a healthy turnover of new cells, and it also supports the structure and tightness of the skin. Further, vitamin E in the shea butter also helps to protect the skin from harmful UV rays of the sun.

In an embodiment, the cocoa extract is a cocoa butter. The cocoa butter is an anti-aging powerhouse, which is high in antioxidants that helps to fight off free-radical damage to the skin. Free radicals can cause skin aging, dark patches, and dull skin. Protecting skin from free-radical damage is a must to maintain healthy and youthful-looking skin. The cocoa butter is also anti-inflammatory, which is another way to help the skin to resist the ravages of time. The cocoa butter is also a deep moisturizer, high in fatty acids. It hydrates the skin on a cellular level, making it a wonderful addition to face moisturizers.

In an embodiment, the glycerin extract is a vegetable glycerin. The vegetable glycerin is a natural humectant. When used with other natural oils and moisturizing ingredients, it seals in moisture on the top layer of the skin while the other ingredients work their way deep into skin cells to provide long-lasting moisture.

In an embodiment, the squalane extract is a squalene oil. The squalane is a natural substance produced in human body. At birth, 12% of human skin surface is made of squalane. But in mid-to-late twenties, its production begins to rapidly decline, making it important to find topical remedies to replace the lost squalane. The squalane oil fights off skin-damaging free radicals that contribute to the signs of aging. It balances oil production, providing enough moisture to keep the skin clear and healthy. Squalane oil also has powerful anti-aging properties and would help the skin age gracefully as well as anti-inflammatory benefits to help with skin conditions such as acne and eczema. Lastly, Squalane oil helps boost blood circulation, which promotes the formation of collagen for firmer, plumper skin. Because its molecular structure is so close to the human skin, squalane oil penetrates deep into the pores where it replenishes skin surface at a cellular level.

In an embodiment, the copaiba extract is a copaiba oil. One of the most notable and popular health benefits of copaiba essential oil is derived from its astringent property. The copaiba's active components tighten the skin, which could help the skin look younger, reduce the appearance of wrinkles, and even strengthen the skin to prevent lesions or wounds as the body ages. Additionally, a study published in Alternative Medicine Review, a journal of clinical therapeutics, reveals that a topical application of copaiba essential oil helps to reduce the appearance of acne on the face.

In an embodiment, the frankincense extract is a frankincense oil. The frankincense strengthens skin and improves its tone, elasticity, and tightness, to naturally slow the signs of aging. The frankincense helps reduce the appearance of large pores and prevent wrinkles. The frankincense also acts as a defense mechanism against facial bacteria, acne and other blemishes. The frankincense oil is also a powerful astringent that helps to lift and tighten the skin, while protecting skin cells.

In an embodiment, the helichrysum extract is a helichrysum oil. The helichrysum is best known for its restorative properties to the skin. Also referred to as the Everlasting or Immortal Flower, helichrysum is used in anti-aging products for its rejuvenating benefits to the skin. helichrysum also helps to reduce the appearance of blemishes.

In an embodiment, the lavender extract is a lavender oil. The lavender is a potent anti-inflammatory that is used to reduce the appearance of skin imperfections. Lavender oil is also an ultra-soothing moisturizer that has a therapeutic scent. It is also an antibacterial that kills bacteria in the pores of skin. The lavender oil also prevents and heals acne.

In an embodiment, the sandalwood extract is a sandalwood oil. The sandalwood extract is high in antioxidants that helps to reduce damage caused by free radicals, exfoliate the skin, soothe sunburns, remove suntan, and reduce the signs of aging including dry skin and wrinkles.

In one embodiment, the skin care composition comprises 70% to 75% of shea extract, 10%-15% of cocoa extract, 10% to 55% of glycerin extract, 0.40% to 0.45%of squalane extract, 0.40% to 0.45% of copaiba extract, 0.40% to 0.45% of frankincense extract, 0.40% to 0.45% of helichrysum extract, 0.40% to 3% of lavender extract, and 25% to 100% of sandalwood extract.

In another embodiment, the skin care composition comprises 72.84% of shea extract, 12.35% of cocoa extract, 12.35% to 24.69% of glycerin extract, 0.41% of squalane extract, 0.41% of copaiba extract, 0.41% of frankincense extract, 0.41% of helichrysum extract, 0.41% to 2.47% of lavender extract, and 27.16% to 100% of sandalwood extract.

In yet another embodiment, the skin care composition comprises 177 ml of shea extract, 30 ml of cocoa extract, 30 ml of glycerin extract, 1 ml of squalane extract, 1 ml of copaiba extract, 1 ml of frankincense extract, 1 ml of helichrysum extract, 1 ml of lavender extract, and 243 ml of sandalwood extract.

In yet another embodiment, the skin care composition comprises ⅕^{th}-⅛^{th} cup of shea extract, ½^{th}-¾^{th} cup of cocoa extract, 1-2 tablespoons of glycerin extract, 15-25 drops of squalane extract, 15-25 drops of copaiba extract, 15-25 drops of frankincense extract, 15-25 drops of helichrysum extract, 15-25 drops of lavender extract, and 15-25 drops of sandalwood extract.

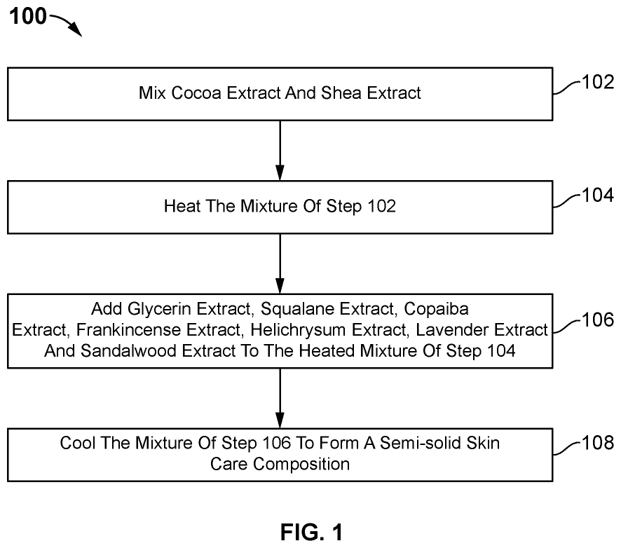

Referring to FIG. 1, in an embodiment, a method of preparing the composition is disclosed. At step 102, the cocoa extract and shea extract are mixed. At step 104, the mixture of the cocoa extract and shea extract are heated to a certain temperature. At step 106, one or more ingredients are added to the heated the cocoa extract and shea extract to form a mixture, wherein the ingredients include glycerin extract, squalane extract, copaiba extract, frankincense extract, helichrysum extract, lavender extract and sandalwood extract. In one embodiment, the ingredients are added at a quantity till the mixture reaches a particular consistency or viscosity. At step 108, the mixture is cooled to form the semi-solid skin care composition. Then, the semi-solid skin care composition could be packed, stored and utilized as a moisture cream. The semi-solid composition changes into a thick liquid on application to the skin surface due to the skin temperature. The solid to liquid phase change enables the composition to adhere to the skin for long duration producing the desired or excellent result.

The present invention does not comprise formaldehyde, artificial fragrances, propylene glycol, hydroquinone, Sodium Lauryl Sulfate (SLS), parabens, preservatives, fillers, dyes. The artificial fragrances are toxic petroleum solvents. These solvents bind various organic molecules and house fragrances. These solvents not only irritate eyes and skin, but they have also been linked to reproductive damage, organ toxicity, muscle control and memory loss. Propylene glycol (PG), or Butylene Glycol, Propylene Glycol is used in facial creams to help bind water and oil. However, the Environmental Protection Agency (EPA) has concluded that the brain, liver and kidneys are all affected by absorption of this toxin through the skin. Hydroquinone also used under the name, tocopherol acetate, has been linked to immunotoxicity, organ system toxicity and cancer. Sodium Lauryl Sulfate's primary use is to create excess foaming in facial cleansers, but it causes stomach issues, redness, pain, sore throat, cough and is also used in several pesticides. Parabens are used as a preservative to lengthen the shelf life of skincare products. Parabens have been linked to an increased risk of breast cancer, fertility issues, hormonal changes such as early menopause for women and early puberty in children.

Advantageously, the composition of the present invention does not utilize any artificial ingredients including, but not limited to, preservatives, binders and fillers. Existing composition utilize binders and artificial ingredients to bind oil and water. However, the present invention utilizes ingredients in oil form. Further, the ratio of ingredients of the present invention eliminates the need to use artificial ingredients. The ratio of composition of the present invention provides the semi-solid skin care composition to adhere to the skin for long duration to produce the desired result. The present invention comprises only active ingredients that works together to provide the desired result. The present invention provides a supple, soft, and younger looking skin naturally, without any artificial or synthetic ingredients, including binders, fillers, dyes, parabens, fragrances or other harmful substances.

Although a single embodiment of the invention has been illustrated in the accompanying drawings and described in the above detailed description, it will be understood that the invention is not limited to the embodiment developed herein, but is capable of numerous rearrangements, modifications, substitutions of parts and elements without departing from the spirit and scope of the invention.

The foregoing description comprises illustrative embodiments of the present invention. Having thus described exemplary embodiments of the present invention, it should be noted by those skilled in the art that the within disclosures are exemplary only, and that various other alternatives, adaptations, and modifications may be made within the scope of the present invention. Merely listing or numbering the steps of a method in a certain order does not constitute any limitation on the order of the steps of that method. Many modifications and other embodiments of the invention will come to mind to one skilled in the art to which this invention pertains having the benefit of the teachings presented in the foregoing descriptions. Although specific terms may be employed herein, they are used only in generic and descriptive sense and not for purposes of limitation. Accordingly, the present invention is not limited to the specific embodiments illustrated herein.

What is claimed is:

1. A skin care composition, comprising:
70% to 75% of shea extract;
10% to 15% of cocoa extract;
10% to 55% of glycerin extract;
0.40% to 0.45% of squalane extract;
0.40% to 0.45% of copaiba extract;
0.40% to 0.45% of frankincense extract;
0.40% to 0.45% of helichrysum extract;
0.40% to 3% of lavender extract, and
25% to 100% of sandalwood extract.

2. The composition of claim 1, comprising:
72.84% of shea extract;
12.35% of cocoa extract;
12.35% to 24.69% of glycerin extract;
0.41% of squalane extract;
0.41% of copaiba extract;
0.41% of frankincense extract;
0.41% of helichrysum extract;
0.41% to 2.47% of lavender extract, and
27.16% to 100% of sandalwood extract.

3. The composition of claim 1, wherein the shea extract is a shea butter.

4. The composition of claim 1, wherein the cocoa extract is a cocoa butter.

5. The composition of claim 1, wherein the glycerin is a vegetable glycerin.

6. The composition of claim 1, wherein the squalane extract is a squalane oil.

7. The composition of claim 1, wherein the copaiba extract is a copaiba oil.

8. The composition of claim 1, wherein the frankincense extract is a frankincense oil.

9. The composition of claim 1, wherein the helichrysum extract is a helichrysum oil.

10. The composition of claim 1, wherein the lavender extract is a lavender oil.

11. The composition of claim 1, wherein the sandalwood extract is a sandalwood oil.

12. The composition of claim 1, is in semi-solid form.

13. The composition of claim 1, liquifies upon application to a skin surface, at the skin temperature.

14. A method for preparing a skin care composition, comprising the steps of:
mixing a cocoa extract and a shea extract;
heating the mixed cocoa and shea extract;
adding a glycerin extract, a squalane extract, a copaiba extract, a frankincense extract, a helichrysum extract, a lavender extract and a sandalwood extract to the heated cocoa and shea extract to form a mixture, and
cooling the mixture to form the semi-solid skin care composition.

15. The method of claim 14, comprising:
70% to 75% of shea extract;
10% to 15% of cocoa extract;
10% to 55% of glycerin extract;
0.40% to 0.45% of squalane extract;
0.40% to 0.45% of copaiba extract;
0.40% to 0.45% of frankincense extract;
0.40% to 0.45% of helichrysum extract;
0.40% to 3% of lavender extract, and
25% to 100% of sandalwood extract.

16. The method of claim 14, comprising:
72.84% of shea extract about;
12.35% of cocoa extract;
12.35% to 24.69% of glycerin extract;
0.41% of squalane extract;
0.41% of copaiba extract;
0.41% of frankincense extract;
0.41% of helichrysum extract;
0.41% to 2.47% of lavender extract, and
27.16% to 100% of sandalwood extract.
